(19) United States
(12) Patent Application Publication
(10) Pub. No.: US 2014/0058247 A1
(43) Pub. Date: Feb. 27, 2014

(54) INFORMATION OBTAINING METHOD AND EVALUATION METHOD

(71) Applicant: Canon Kabushiki Kaisha, Tokyo (JP)

(72) Inventors: Yasuhiro Kawashima, Tokyo (JP); Jungo Miyazaki, Kawasaki-shi (JP); Yoshikatsu Ichimura, Tokyo (JP)

(21) Appl. No.: 13/967,268

(22) Filed: Aug. 14, 2013

(30) Foreign Application Priority Data
Aug. 22, 2012 (JP) 2012-182962

(51) Int. Cl. A61B 5/0484

(52) U.S. Cl. CPC A61B 5/04842
USPC 600/411; 600/544; 600/410

(57) ABSTRACT

An information obtaining method includes (1) presenting a first visual stimulus to a living body, (2) obtaining brain activity data in the primary visual cortex and a color vision related area during presentation of the first visual stimulus, (3) presenting a second visual stimulus different from the first visual stimulus to the living body, (4) obtaining brain activity data in the primary visual cortex and the color vision related area during presentation of the second visual stimulus, and (5) obtaining brain activity information on the basis of the ratio between the brain activity data in the primary visual cortex, obtained in (2), and the brain activity data in the primary visual cortex, obtained in (4), and the ratio between the brain activity data in the color vision related area, obtained in (2), and the brain activity data in the color vision related area, obtained in (4).

### BACKGROUND

1. Field

Aspects of the present invention generally relate to an information obtaining method of obtaining brain activity information from a living body to which a visual stimulus is given, and to an evaluation method.

2. Description of the Related Art

In evaluation of how a human being (human body) recognizes the colors, shape, or material of an object when he or she perceives the same, techniques based on subjectivity such as questionnaires are generally used. However, such evaluation techniques and the results obtained thereby have difficulty in performing quantitative representation.

Since the subjectivity of a human being is vague as described above, various methods have been devised to enhance the confidence of data.

Here, in Japanese Patent Laid-Open No. 5-89240, as a method of quantitatively evaluating the visibility of a color displayed on a display, a human being's psychological and physiological data in response to presentation of a stimulating color is measured.

Specifically, psychological evaluation is conducted on the visibility of displayed color characters, and, at the same time, physiological evaluation is conducted on brain waves and eyeball movement. Accordingly, the visibility of a color is evaluated from both sides, namely, subjectivity and objectivity.

The present inventors have discovered the following in the method described in Japanese Patent Laid-Open No. 5-89240.

That is, in the method described in Japanese Patent Laid-Open No. 5-89240, because the visibility of a color is evaluated on the basis of a combination of psychological data and physiological data obtained by measurement of brain waves, it is difficult to eliminate the influence of a human being's subjectivity included in the evaluation result. Thus, it has been difficult to perform objective evaluation of the visibility of a color.

In accordance with such circumstances, there has been a demand for a more accurate method of obtaining information relating to a human being's perception.

### SUMMARY

Aspects of the present invention generally provide an information obtaining method and an evaluation method capable of obtaining or evaluating highly accurate information when obtaining or evaluating brain activity information from a living body to which a visual stimulus is given.

According to an aspect of the present invention, an information obtaining method of obtaining brain activity information from a living body to which different visual stimuli are given, includes

(1) presenting a first visual stimulus to the living body,
(2) obtaining brain activity data in the primary visual cortex of the living body and brain activity data in a color vision related area of the living body during presentation of the first visual stimulus,
(3) presenting a second visual stimulus different from the first visual stimulus to the living body,
(4) obtaining brain activity data in the primary visual cortex and brain activity data in the color vision related area during presentation of the second visual stimulus, and
(5) obtaining brain activity information on the basis of the ratio (ratio 3) between the ratio between the brain activity data in the primary visual cortex, which is obtained in (2), and the brain activity data in the primary visual cortex, which is obtained in (4) (ratio 1), and the ratio between the brain activity data in the color vision related area, which is obtained in (2), and the brain activity data in the color vision related area, which is obtained in (4) (ratio 2). The ratio 3 is ratio 2/ratio 1.

According to the present disclosure, an information obtaining method and an evaluation method capable of obtaining or evaluating highly accurate information can be provided.

Further features of the present disclosure will become apparent from the following description of exemplary embodiments with reference to the attached drawings.

### BRIEF DESCRIPTION OF THE DRAWINGS

FIG. 1 is a diagram for describing an information obtaining method according to an exemplary embodiment.

FIGS. 2A to 2C are diagrams for describing the visual cortex in the information obtaining method according to the exemplary embodiment.

FIG. 3 is a diagram for describing the method of fMRI, which is a brain activity measuring method used in the exemplary embodiment.

FIGS. 4A and 4B are diagrams for describing the method of obtaining amplitude values from time-series changes in brain activity data used in the exemplary embodiment.

FIG. 5 is a diagram showing amplitude values in the primary visual cortex and a color vision related area when a color printed matter is presented to a human being under illuminance conditions in the exemplary embodiment.

FIG. 6 is a diagram for describing a measuring technique used in an example of the exemplary embodiment.

FIG. 7 is a diagram for describing a sample holding unit used in the example of the exemplary embodiment.

FIG. 8 is a diagram for describing the steps of a brain activity measuring method used in the example of the exemplary embodiment.

FIG. 9 is a diagram showing amplitude values of brain activity data under a low illuminance condition and a high illuminance condition in each visual cortical area, serving as an example of brain activity data obtained in the example of the exemplary embodiment.

### DESCRIPTION OF THE EMBODIMENTS

Exemplary embodiments will be described below. These embodiments are not seen to be limiting.

To begin with, an information obtaining method according to the embodiment will be described.

The information obtaining method according to the embodiment is an information obtaining method of obtaining brain activity information from a living body to which a visual stimulus is given, including the following (1) to (4) steps:

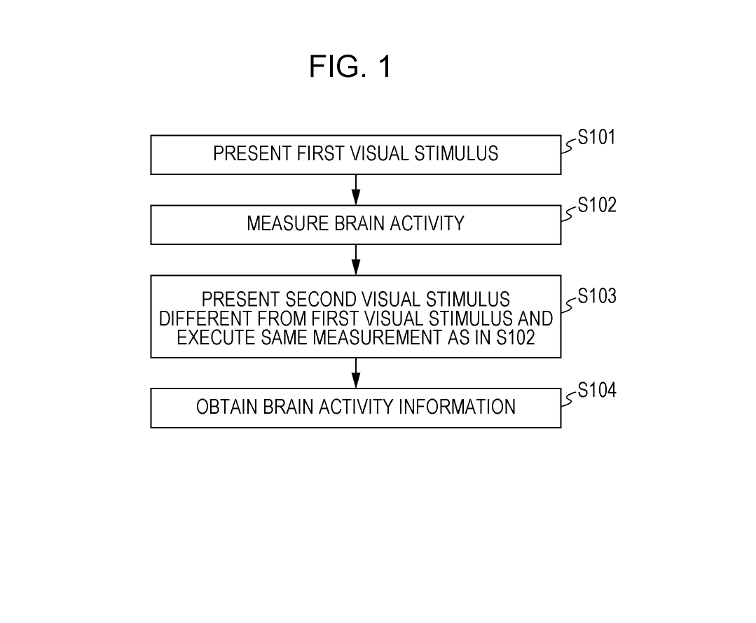

(1) presenting a first visual stimulus to a living body, as shown in step S101 of FIG. 1;
(2) measuring brain activity in the primary visual cortex and a color vision related area of the living body during presentation of the first visual stimulus, as shown in step S102 of FIG. 1;
(3) presenting a second visual stimulus different from the first visual stimulus to the living body and executing the same step as (2), as shown in step S103 of FIG. 1; and
(4) calculating the ratio of brain activity data in the primary visual cortex and the ratio of brain activity data in the color vision related area from the measurement results obtained in (2) and (3) described above, and obtaining brain activity information on the basis of the ratio between the ratio in the color vision related area and the ratio in the primary visual cortex, as shown in step S104 of FIG. 1.

Next, the relationship among these steps will be further described.

In step (1), a visual stimulus is presented to a living body (such as a human living body) by presenting a printed matter, a real object, a video image, or the like.

In step (2), during presentation of the visual stimulus in step (1), brain activity in the primary visual cortex and a color vision related area is measured. Brain activity can be quantified by measuring an action potential of the brain's nerve cells, or an electromagnetic field or cerebral blood flow caused by the action potential.

Measurement of brain activity during presentation of a visual stimulus means continuously or intermittently obtaining brain activity data induced by presenting the visual stimulus for a certain period from timing at which presentation of the visual stimulus is started. To obtain meaningful information from brain activity data, it is necessary to obtain brain activity data for a certain period in accordance with the brain activity data and the type of apparatus used to measure brain activity data. For example, to measure brain activity as a change in cerebral blood flow relating to metabolic activity of the cerebral nervous system, a change in cerebral blood flow starts responding, with a few seconds delay, the presentation of a visual stimulus, and this response continues for a few seconds. Therefore, it is necessary to obtain brain activity data for at least a few seconds or longer after the start of presentation of a visual stimulus. Needless to say, the longer the period for which brain activity is measured, the greater the amount of brain activity data that can be obtained. However, this also means that the period of time in which a living body, serving as a target of measurement of brain activity, is constrained becomes longer. As a result, the living body becomes tired, and the risk of an artifact being mixed in the obtained brain activity data becomes higher. Therefore, the period for measuring brain activity, which is a period that is sufficient for obtaining desired information and that will not exhaust a living body, serving as a target of measurement of brain activity, is preferably a period of about a few seconds to a few tens of minutes.

In step (3), in presentation of a visual stimulus different from that in step (1) to the human body, a visual stimulus itself may be changed from that in step (1) and may be presented to the human body, or the same visual stimulus as that in step (1) may be presented to the human body in a different environment from that in step (1).

For example, in a lighting environment where the human body sees the visual stimulus, while properties such as the chromaticity, color temperature, and irradiation light wavelength of a lighting apparatus are controlled, only the magnitude of illuminance may be changed, and the same visual stimulus may be presented.

In step (4), the ratios are calculated from brain activity data in the primary visual cortex and the color vision related area, which have been measured in steps (2) and (3), thereby obtaining brain activity information.

Obtaining of brain activity information means, for example, calculation of time-series changes in brain activity from brain activity data in the primary visual cortex and the color vision related area, and obtaining of amplitude values from the waveforms of the time-series changes.

Using the amplitude values, ratios of brain activity data in the individual visual cortical areas relating to different visual stimuli are calculated.

Using the ratio between the ratio in the color vision related area and the ratio in the primary visual cortex as an index, for example, information relating to the visibility of colors when the human body perceives colors is obtained.

When the ratio between the amplitude values in the color vision related area is greater than the ratio between the amplitude values in the primary visual cortex, it can be evaluated that a great change has occurred in the visibility of colors perceived by the human body in response to the changed visual stimulus.

Specifically, it can be evaluated that, relating to a visual stimulus from which a great brain activity response is made, the human body has perceived the colors as colorful or the human body has clearly perceived the brightness of the colors. Amplitude values used to obtain ratios can be quantitatively obtained by using a technique described later.

Using such a method, brain activity in the visual cortex is measured, thereby obtaining brain activity information from a living body to which a visual stimulus is given, such as information relating to the visibility of perceived colors.

The information obtaining method according to the embodiment is a method that is less susceptible to factors other than a visual stimulus.

That is, because this method is less susceptible to the individual's subjectivity, it can be said that the method is a highly accurate method as a method of obtaining information from a living body to which a stimulus is given, such as a method of obtaining perception information relating to the visibility of colors.

Also, the information obtaining method according to the embodiment is a method preferable to be used in evaluation of color characteristics of a visual stimulus or the like.

That is, brain activity information can be obtained from a living body to which a visual stimulus is given, and color characteristics of the visual stimulus can be evaluated from the obtained brain activity information. Therefore, it can be said that this method is a highly accurate method as a visual stimulus evaluation technique.

Also, the information obtaining method according to the embodiment is a method preferable to be used in evaluation of an environment where a visual stimulus is observed.

That is, in obtaining of brain activity information from a living body to which a visual stimulus is given, an observation environment such as brightness is controlled, and, after brain activity information is obtained, the correlation between the obtained brain activity information and the observation environment is examined, thereby evaluating the observation environment. Therefore, it can be said that this method is a highly accurate method as an observation environment evaluation technique.

Any visual stimulus can be used as a visual stimulus in the embodiment as long as it changes the brain activity of a living body.

For example, examples of visual stimuli include a video stimulus projected from a projector or the like, a video stimulus displayed on a display, and a real object stimulus such as a printed matter, a cloth, or a plastic article.

A visual stimulus may be one of a video stimulus and a real object stimulus, or may be a stimulus combining these stimuli. When a visual stimulus is a printed matter, the evaluation method according to the embodiment is preferably a printed matter evaluation method and a printed matter observation environment evaluation technique.

Also, a living body in the embodiment means a human being (human body) or any living body such as a monkey or a cat. In the information obtaining method according to the embodiment, a living body is preferably a human body. Hereinafter, the case in which a living body is a human body will be described. Also, a human body may be referred to as a subject in this Specification.

Brain activity in the embodiment refers to a brain activity response of a living body to which a visual stimulus is given. A brain activity response can be quantified by measuring a change in action potential of the brain's nerve cells, a change in an electromagnetic field caused by a change in action potential, or a change in cerebral blood flow caused by a change in action potential.

The action potential of the brain's nerve cells can be measured by using a neural activity recording apparatus using an electrode. A change in an electromagnetic field caused by a change in action potential can be measured by using an electroencephalograph or a magnetoencephalograph.

A change in cerebral blood flow caused by a change in action potential can be measured by using functional magnetic resonance imaging (fMRI) using a magnetic resonance imaging (MRI) apparatus, a near infrared spectroscopy and imaging (NIRS) apparatus using near infrared (NIR) spectroscopy, or a positron computed tomography measuring apparatus.

Also, brain activity information in the embodiment means brain activity data obtained by measuring the above-described brain activity or information obtained from the data.

Examples of brain activity information include information relating to perception of the visibility of colors, particularly the colorfulness of colors perceived by a living body, or the like.

Information relating to the colorfulness of perceived colors can be obtained from brain activity data such as a change in action potential of the brain's nerve cells, a change in an electromagnetic field caused by a change in action potential, or a change in cerebral blood flow caused by a change in action potential.

The visibility of colors in the embodiment means the perceptual and psychological state of a living body, that is, how a living body grasps the characteristics of colors that change according to a light source or an object.

The characteristics of colors are greatly affected by three properties of colors, that is, hue, saturation, and brightness. Hue means the property of colors perceived as ranging from red through green, and blue. Saturation means the colorfulness of colors. Brightness means brightness of colors.

A color with hue, saturation, and brightness is referred to as a chromatic color. In contrast, a color that only has brightness and has no hue or saturation is referred to as an achromatic color.

White, black, and gray are achromatic colors. A living body may differently perceive the visibility of colors, such as the colorfulness of colors, depending on an environment in which a visual stimulus is observed.

Also, the information obtaining method according to the embodiment preferably obtains information relating to a living body's perception of the visibility of colors.

The information obtaining method according to the embodiment can be preferably used in evaluation of perception of the visibility of colors by a living body who sees a real object such as a printed matter or a video image.

Vision in the embodiment means a sense in response to visible light serving as a physical input. Also, visual information refers to information on the colors, shape, material, movement, texture, depth, or the like of an object in the outside world, information on the category of an object, or spatial information on the outside world such as the positional relationship of an object. The brain's area that is in charge of an initial process of processing visual information is the visual cortex.

In the information obtaining method according to the embodiment, brain activity in the primary visual cortex (V1 (V1d, V1v)) of the visual cortex are measured, and, at the same time, brain activity in visual area V4 (V4d, V4v) or visual area V8, serving as a color vision related area, is preferably measured. The color vision related area is an area considered to be associated with perception of color vision. Besides V4 and V8, the fusiform gyrus, the lingual gyrus, and the collateral sulcus are also known as color vision related areas.

When V4v or V8, which is a color vision related area, serves as a target of measurement, perception of the visibility of colors can be accurately evaluated, which is therefore preferable.

Alternatively, among these color vision related areas, brain activity in an area covering a plurality of areas (such as an area covering V4v and V8) may be measured.

Alternatively, in measurement of brain activity in a particular area, brain activity in a partial area of the particular area may be measured.

Further, in measurement of brain activity in an area covering a plurality of areas, brain activity in partial areas of the plurality of areas (such as a partial area of V4v and a partial area of V8) may be measured.

Note that the visual cortex includes, besides the above-described areas,

visual area V2 (V2d, V2v),
visual area V3 (V3, V3A),
visual area VP (ventral posterior area),
visual area MT (middle temporal area),
visual area MST (middle superior temporal area), visual area V7, visual area LO (lateral occipital area), and so forth. There are other classifications of visual cortical areas corresponding to V4v and V8, and there exists another classification that these areas are hV4, VO1, and VO2.

In the embodiment, these areas are specified by the representations “V4v” and “V8”.

Visual information is mainly processed by the visual cortex of the cerebral cortex, which is recognized in the occipital lobe, part of the parietal lobe, and part of the temporal lobes.

Note that an area located at the front of the cerebral cortex is the frontal lobe, an area located at back is the occipital lobe, an area located at the top is the parietal lobe, and areas located at the sides are the temporal lobes.

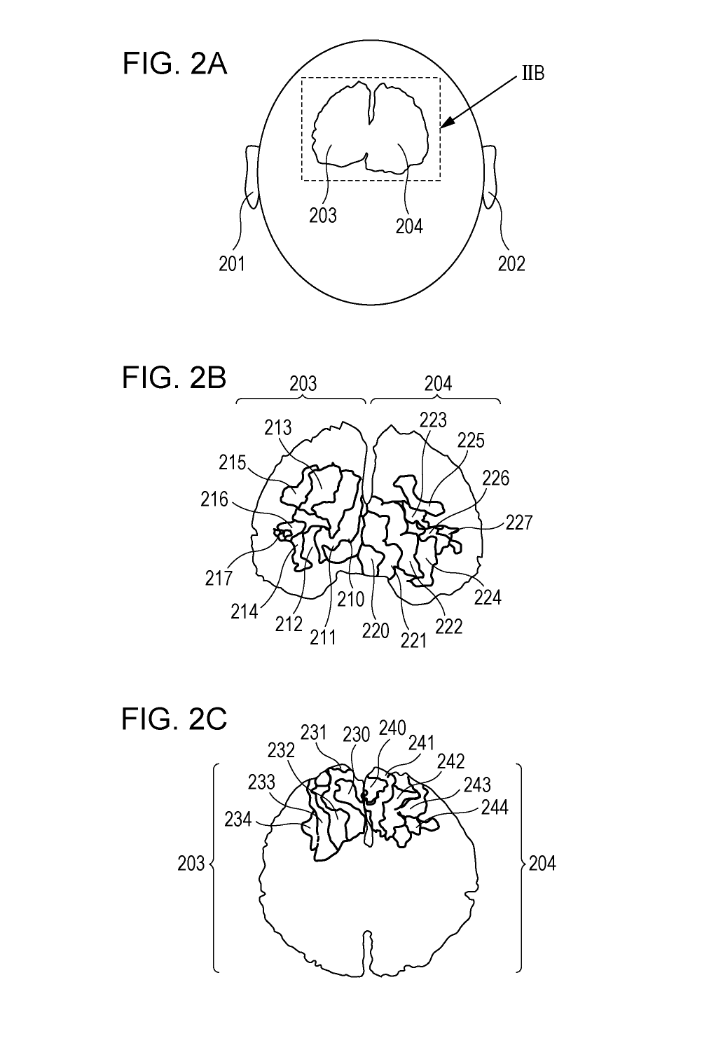

FIG. 2A is a schematic diagram of the cerebral cortex, viewed from the occipital lobe.

FIG. 2B is an enlarged diagram of the cerebral cortex shown in FIG. 2A.

FIG. 2C is a schematic diagram of the enlarged diagram of the cerebral cortex, shown in FIG. 2B, viewed from below. In FIGS. 2A to 2C, the left and right cerebral hemispheres, which are actually connected by the corpus callosum, are shown as being separated, for the sake of convenience.

Note that the schematic diagrams are only exemplary, and the shape and size of the brain and the area division of the visual cortex may be different from one human body to another.

In FIG. 2A, reference numeral 201 represents the left ear, reference numeral 202 represents the right ear, reference numeral 203 represents the left cerebral hemisphere, and reference numeral 204 represents the right cerebral hemisphere.

In the left cerebral hemisphere shown in FIG. 2B, reference numeral 210 represents V1 (V1v, V1d), reference numeral 211 represents V2 (V2v, V2d), reference numeral 212 represents V3, reference numeral 213 represents V3A, reference numeral 214 represents V4 (V4v, V4d), reference numeral 215 represents V7, reference numeral 216 represents visual area LO, and reference numeral 217 represents visual area MT.

Also, in the right cerebral hemisphere 204, reference numeral 220 represents V1 (V1v, V1d), reference numeral 221 represents V2 (V2v, V2d), reference numeral 222 represents V3, reference numeral 223 represents V3A, reference numeral 224 represents V4 (V4v, V4d), reference numeral 225 represents V7, reference numeral 226 represents visual area LO, and reference numeral 227 represents visual area MT.

Also, in the left cerebral hemisphere 203 shown in FIG. 2C, reference numeral 230 represents V1 (V1v, V1d), reference numeral 231 represents V2 (V2v, V2d), reference numeral 232 represents VP, reference numeral 233 represents V4 (V4v, V4d), and reference numeral 234 represents V8. Also, in the right cerebral hemisphere 204, reference numeral 240 represents V1 (V1v, V1d), reference numeral 241 represents V2 (V2v, V2d), reference numeral 242 represents VP, reference numeral 243 represents V4 (V4v, V4d), and reference numeral 244 represents V8.

As shown in FIG. 2B and FIG. 2C, the visual cortical areas exist at substantially symmetrical positions of the left and right hemispheres.

Visual information is first input to V1, located in the posterior end of the occipital lobe of each of the left and right hemispheres. Thereafter, the visual information is processed in visual cortical areas such as V2, V3, and V3A. The visual information processed in the occipital lobe visual cortical areas is sequentially transferred to the visual association areas in the parietal lobe, temporal lobes, and frontal lobe, and integrated to give rise to a visual function such as visual perception or visual memory. In Broadmann areas, V1 is located in Broadmann area 17, V2 is located in Broadmann area 18, and V3 is located in Broadmann area 19.

In the visual cortex of the cerebral cortex, visual information processing after V1 and V2 is performed in two primary pathways.

One is called the dorsal visual stream, which is a visual information processing pathway that passes through areas at the back of the cerebral cortex, such as V3, V3A, visual area LO, and visual area MT.

Many nerve cells that respond to visual motions or binocular parallax exist in the dorsal visual stream, and they are considered to contribute to spatial awareness of objects including himself/herself and perception of motion states.

The other one is called the ventral visual stream, which is a visual information processing pathway that passes through areas in the ventral part of the cerebral cortex, such as V4v and V8.

Many nerve cells that respond to colors and shapes exist in the ventral visual stream, and they are considered to contribute to recognition of objects by vision.

When a color is perceived, a change occurs in a brain activity response in the visual cortex of the ventral part of the cerebral cortex.

That is, when a living body is perceiving a color, a great response change occurs in brain activity in the color vision related area of the ventral part.

Therefore, a response in the color vision related area is compared with reference to a response at the same time in the primary visual cortex, thereby obtaining information relating to perception of the visibility of colors.

Specifically, information relating to perception of the visibility of colors or the like can be obtained by using, as indices, the ratios between brain activity changes in the primary visual cortex and in the color vision related area when two different visual stimuli are given to a living body.

To obtain perception information relating to a living body's visibility of colors, the information obtaining method according to the embodiment preferably measures brain activity not only in the above-described visual cortical areas but also in the fusiform gyrus and collateral sulcus of the ventral stream.

Since the fusiform gyrus and collateral sulcus are also part of the color vision related area, more detailed information relating to perception of the visibility of colors can be obtained by comparing brain activity responses in these areas with reference to a brain activity response in the primary visual cortex.

To obtain perception information relating to the visibility of colors, brain activity responses to be measured are not limited to those in the above-described areas, but brain activity responses in any area of the cerebral cortex can be used.

Also, brain activity responses only in the right hemisphere, brain activity responses only in the left hemisphere, or averaged responses of brain activity responses in the left and right hemispheres may be used.

A brain activity response of a living body to which a visual stimulus is given can be quantified by measuring a change in action potential of the brain's nerve cells or a change in an electromagnetic field or a change in cerebral blood flow caused by a change in action potential.

Hereinafter, as an exemplary method of measuring brain activity, the method of fMRI will be described in detail.

The method of fMRI is a method of visualizing a hemodynamic response related to a change in activity of the brain's nerve cells or the like by using an MRI apparatus.

An MRI apparatus is an apparatus configured to obtain an image by applying a static magnetic field to a to-be-measured portion of a subject, further applying a particular high frequency magnetic field, and utilizing nuclear magnetic resonance caused by application of these magnetic fields.

Nerve cells consume oxygen in an activity change and temporarily enter an oxygen-depleted state. To avoid such depletion, blood is sent to cerebral blood vessels near these nerve cells, and the nerve cells are refilled with oxygen.

The method of fMRI is a brain activity measuring method for measuring the amount of change in oxyhemoglobin or deoxyhemoglobin included in the cerebral blood flow by using an MRI apparatus.

To measure the brain hemodynamics by using the method of fMRI, a portion responding to a visual stimulus is specified by performing statistic processing, and that portion is displayed as a color map on an anatomical image, thereby visualizing brain activity.

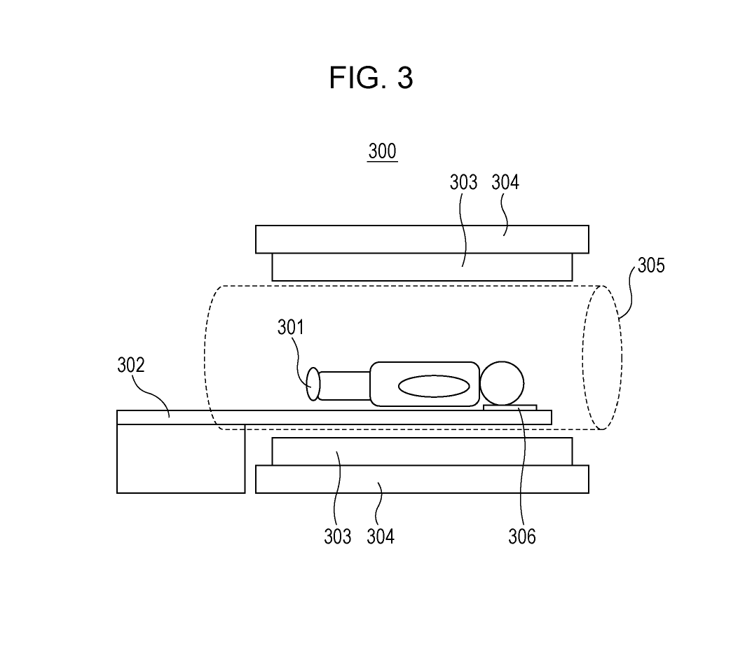

Measurement by using the method of fMRI is preferably performed by an apparatus configuration such as that shown in FIG. 3. In FIG. 3, reference numeral 300 represents an MRI apparatus, reference numeral 301 represents a subject, reference numeral 302 represents a bed of the MRI apparatus 300, reference numeral 303 represents a gradient magnetic field coil, reference numeral 304 represents a superconducting magnet, and reference numeral 305 represents a bore.

In the above-described configuration, the subject 301 lies down in the interior of the bore 305. An MR signal detecting coil 306 is placed on the head of the subject 301 to detect an electromagnetic signal generated by a change in cerebral blood flow involved in neural activity of the subject 301.

To ensure the visual field in front of the subject 301 and to perform highly sensitive brain activity measurement of the visual cortex of the cerebral cortex, a surface coil-type radio frequency coil is preferably used as the coil 306.

Brain activity information is obtained by performing the above-described brain activity measurement. Alternatively, brain activity information is obtained by analyzing brain activity data obtained by performing the brain activity measurement. The information obtaining method according to the embodiment preferably obtains brain activity information relating to the visibility of colors from luminance information of captured fMRI images.

Hereinafter, the method of evaluating the visibility of colors will be described.

Brain activity information obtained by the information obtaining method according to the embodiment may be information relating to perception of the visibility of colors, particularly information relating to the colorfulness of perceived colors.

By obtaining information relating to the colorfulness of perceived colors, how a human body perceives characteristics of colors in response to a visual stimulus can be evaluated.

The method of evaluating perception of the visibility of colors is performed on the basis of brain activity data in a state where a visual stimulus is given to a human body.

For example, the method of obtaining brain activity information when a color printed matter is used as a visual stimulus under different illuminance conditions will be described as below.

Time-series changes in brain activity data in the primary visual cortex and the color vision related area when a color printed matter, irradiated with light with a first illuminance, is presented to a human body are calculated.

Similarly, time-series changes in brain activity data in the primary visual cortex and the color vision related area when the color printed matter, irradiated with light with a second illuminance, is presented to the human body are calculated.

Amplitude values are obtained from the waveforms of the time-series changes, and, using the amplitude values, the ratios between the brain activity data obtained in the visual cortical areas with the first illuminance light and the brain activity data obtained in the visual cortical areas with the second illuminance light are calculated. Finally, the ratio between the ratio in the color vision related area and the ratio in the primary visual cortex serves as an index, and the visibility of colors is evaluated.

As described above, information relating to perception of the visibility of colors can be quantified by using the ratios of the amplitude values.

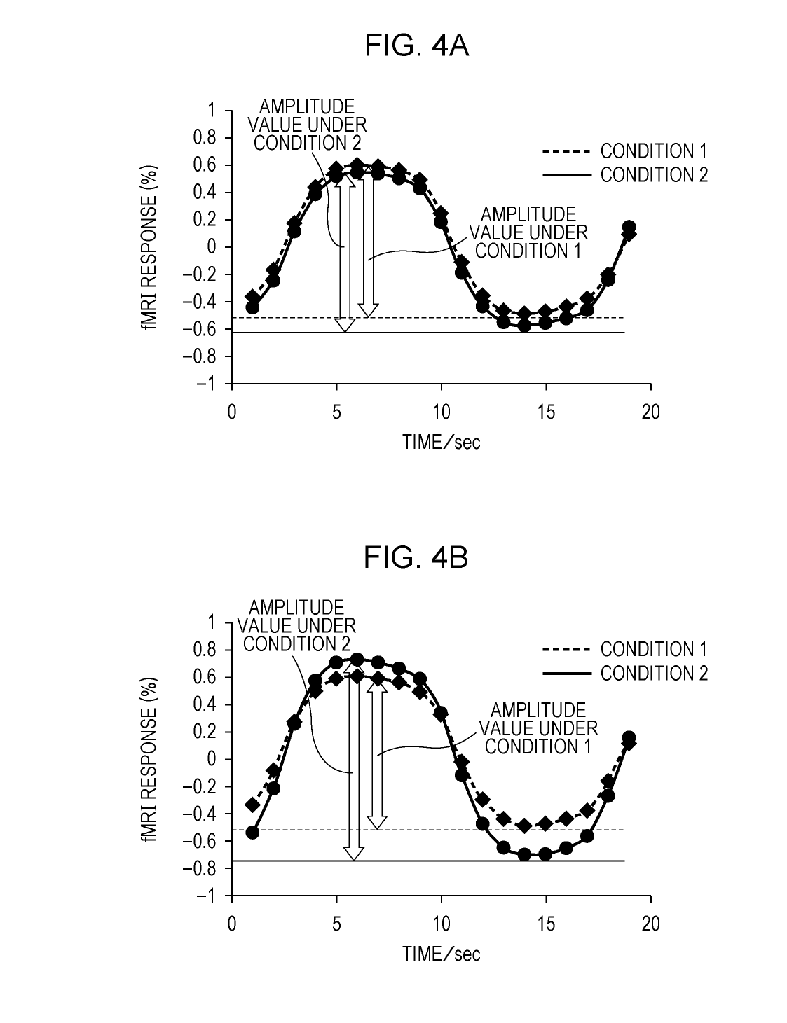

FIGS. 4A and 4B are graphs showing a method of obtaining amplitude values from time-series changes in brain activity data. FIGS. 4A and 4B show time-series changes in brain activity data in the primary visual cortex and the color vision related area under two illuminance conditions, measured by the method of fMRI.

As shown in the graph of FIG. 4A, amplitude values are calculated from waveforms under the conditions, and the ratio between the amplitude values is obtained, thereby calculating the amount of change in brain activity data in the primary visual cortex. Similarly, the ratio in the color vision related area is calculated (FIG. 4B). The ratio in the color vision related area is divided by the ratio in the primary visual cortex, thereby calculating a ratio serving as an index of brain activity information.

That is, in the information obtaining method according to the embodiment, an exemplary method of obtaining the ratio of amplitude values from time-series changes in brain activity data is as follows.

(i) Time-series changes are calculated from brain activity data in the primary visual cortex obtained in steps (2) and (3) described above, and amplitude values are obtained from the waveforms of the time-series changes. The ratio between the obtained two amplitude values is calculated, which serves as the amount of change in brain activity data in the primary visual cortex.

(ii) The same steps as (i) are executed for brain activity data in the color vision related area, which is obtained in steps (2) and (3) described above.

(iii) The ratio between the ratios obtained in (i) and (ii) described above is further calculated. In this calculation, the ratio in the color vision related area is divided by the ratio in the primary visual cortex, thereby calculating a ratio serving as an index.

When a color printed matter is used as a visual stimulus, if the finally obtained ratio is near 1, it means that no big difference has occurred in a brain activity change between the primary visual cortex and the color vision related area of the subject. Therefore, it means that no big difference has also occurred in brain activity relating to color perception involved in a change in illuminance of light emitted to the visual stimulus.

On the contrary, if the ratio is greater than 1, it means that a brain activity change in the color vision related area is greater than that in the primary visual cortex. It can thus be understood that a significant difference has occurred in a brain activity response relating to color perception involved in a change in illuminance.

That is, it can be evaluated that, under the illuminance condition where the subject has shown a greater response in the color vision related area, the subject perceives the colors of the color printed matter, which is the visual stimulus, to be colorful, or the subject clearly perceives the brightness of the colors of the printed matter.

To obtain perception information relating to a living body's visibility of colors, the information obtaining method according to the embodiment preferably includes, after step (1) described above and before step (3) described above, the step of presenting, for example, a grayscale printed matter, which is generated by removing hue and saturation from the color printed matter, to the living body.

By comparing brain activity in response to presentation of the grayscale printed matter with brain activity in response to presentation of the color printed matter, a brain activity response corresponding to the colors of a target printed matter can be more accurately obtained.

Also, to obtain perception information relating to a living body's visibility of colors, the information obtaining method according to the embodiment preferably includes, after step (1) described above and before step (3) described above, the step of returning brain activity in the primary visual cortex and the color vision related area of the living body to a steady state.

Specifically, for example, it is preferable to present a gray control stimulus to the living body. The gray control stimulus is preferably generated with the gray color with an average luminance calculated from the luminances of the grayscale printed matter.

By presenting the control stimulus to the living body for a certain time, brain activity in the visual cortex can be controlled to be a state prior to presentation of the first visual stimulus, that is, to a steady state. The steady state means a state in which the action potential of the nerve cells of the brain of a human body and the cerebral blood flow caused by a change in action potential become stable as in a rest state, and this stable state is maintained.

By having the step of returning brain activity to a steady state, time-series changes in brain activity data in response to presentation of a visual stimulus can be more accurately obtained, and more accurate amplitude values can be obtained.

As described above, a subject's perception of the visibility of colors in response to a visual stimulus can be evaluated on the basis of brain activity information obtained by the information obtaining method according to the embodiment.

Therefore, according to the information obtaining method according to the embodiment, if the finally obtained ratio is great, it can be evaluated that, for example, the subject recognizes that the colors of the visual stimulus to which a great brain activity response has been given are colorful, or the subject clearly perceives the brightness of the colors.

For example, the following evaluation can be performed. Brain activity data in the primary visual cortex and in the color vision related area in the case where a color printed matter is used as a visual stimulus is measured.

In measurement of the brain activity, two illuminance conditions are set. For the obtained brain activity data, ratios are calculated by performing steps (i) to (iii) described above.

Ratios are obtained for a plurality of subjects. The average of the obtained ratios or the minimum ratio is preferably set as a threshold.

For example, if the finally obtained ratio is greater than the threshold, it can be evaluated that the colors are colorfully seen. Needless to say, the threshold setting method is not limited to that described above.

In determination of the threshold, it is desirable to apply in advance the information obtaining method according to the embodiment to an arbitrary subject and to clarify the relationship between the obtained ratio and perception of the visibility of colors.

For example, a color printed matter is presented to the subject under different illuminance conditions (first condition and second condition), and brain activity in the primary visual cortex and the color vision related area of the subject while looking at the color printed matter under these illuminance conditions is measured.

At this time, it is preferable to use a grayscale printed matter as a visual stimulus that serves as a reference, besides the color printed matter.

Also, it is preferable to use a gray control stimulus for returning brain activity to a steady state.

The subject is given visual stimuli in, for example, such a order as: the color printed matter, followed by the gray control stimulus, followed by the grayscale printed matter, followed by the gray control stimulus, followed by the color printed matter, and finished by the gray control stimulus.

From the measured and obtained brain activity information, the amplitude values of time-series changes in brain activity data in the visual cortical areas when the subject sees the color printed matter under the first condition and the second condition are calculated.

Also, whether there is a significant difference between brain activity data when the subject sees the color printed matter under the first condition and the second condition is statistically tested, and a threshold for determining whether the subject (living body) recognizes that the colors of the color printed matter are colorful is calculated.

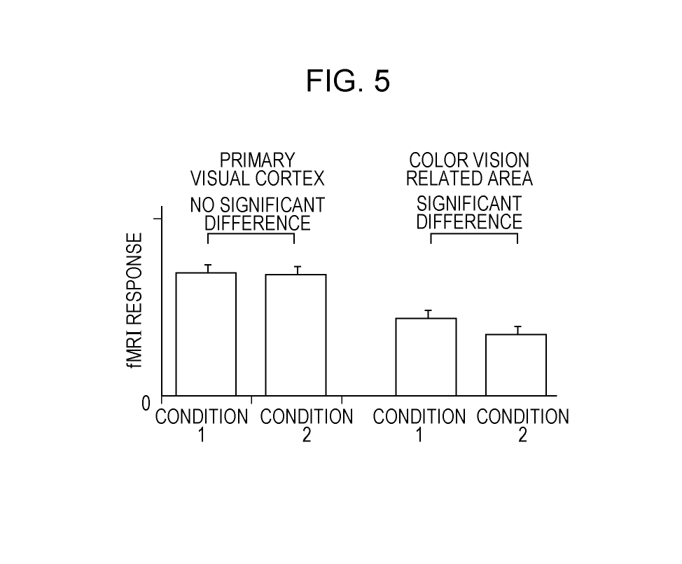

The graph shown in FIG. 5 is a bar graph representing amplitude values in the primary visual cortex and the color vision related area when a color printed matter is presented to a human body under two illuminance conditions.

A t-test is conducted to statistically determine significant data in data represented in FIG. 5.

A t-test is a parametric technique for testing a statistically significant difference, which is used to test, assuming that two populations to be compared have normal distribution, whether the averages of the two populations are equivalent.

The result in FIG. 5 confirms that there is a significant difference, depending on illuminance conditions, in the color vision related area although no significant difference is confirmed in the primary visual cortex.

The case shown in FIG. 5 is only exemplary, and any method may be used as long as it can clarify the corresponding relationship between perception relating to the visibility of colors and the subject's brain activity and determine a ratio threshold.

Next, the evaluation method according to the exemplary embodiment.

The evaluation method according to the embodiment is a perception evaluation method of evaluating which of two objects, presented to a subject, has colors that are perceived by the subject to be more colorful than the other, and has the following (1) to (5) steps:

(1) presenting a first object to the subject;
(2) obtaining an fMRI image of the primary visual cortex and an fMRI image of visual area V4 or visual area V8 of the subject during presentation of the first object;
(3) presenting a second object different from the first object to the subject;
(4) obtaining an fMRI image of the primary visual cortex and an fMRI image of visual area V4 or visual area V8 during presentation of the second object; and
(5) evaluating that the subject is perceiving colors of the second object to be more colorful when luminance of the fMRI image of the primary visual cortex, which is obtained in step (4) described above, is within a range from greater than or equal to 0.9 times to less than or equal to 1.1 times luminance of the fMRI image of the primary visual cortex, which is obtained in step (2) described above, and when luminance of the fMRI image of visual area V4 or visual area V8, which is obtained in step (4) described above, is greater than or equal to 1.2 times luminance of the fMRI image of visual area V4 or visual area V8, which is obtained in step (2) described above.

In the embodiment, an evaluation program for causing steps including steps (1) to (5) described above to be executed or an information obtaining program for causing steps including steps (1) to (5) of the above-described information obtaining method of the embodiment to be executed can be configured.

These programs according to the embodiment may be recorded on recording media or may be downloaded from the Internet. The programs can be read by a computer.

In the embodiment, a computer-readable recording medium having recorded thereon the above-described information obtaining program or the evaluation program can be configured.

That is, a computer-readable recording medium having recorded thereon a program for causing steps including steps (1) to (5) of the above-described evaluation method or the previously-described information obtaining method of the embodiment to be executed can be configured.

Here, examples of recording media include compact discs (CDs) (CD-recordable (CDR), CD-rewritable (CDRW), etc.), digital versatile discs (DVDs) (DVD-recordable (DVDR), DVD-rewritable (DVDRW)), flash memories, hard disks, magnetic tapes, floppy (registered trademark) disks, and the like.

### EXAMPLES

Hereinafter, examples of the exemplary embodiment will be described.

### First Example

In a first example, brain activity in the primary visual cortex and visual area V8, which is the color vision related area, when a color printed matter is presented to a subject under different illuminance conditions was measured, and, using the obtained brain activity data, the subject's perception of the visibility of colors of the color printed matter was evaluated.

In this example, an MRI apparatus was used as a brain activity measuring apparatus, and the method of fMRI was used as a measurement technique.

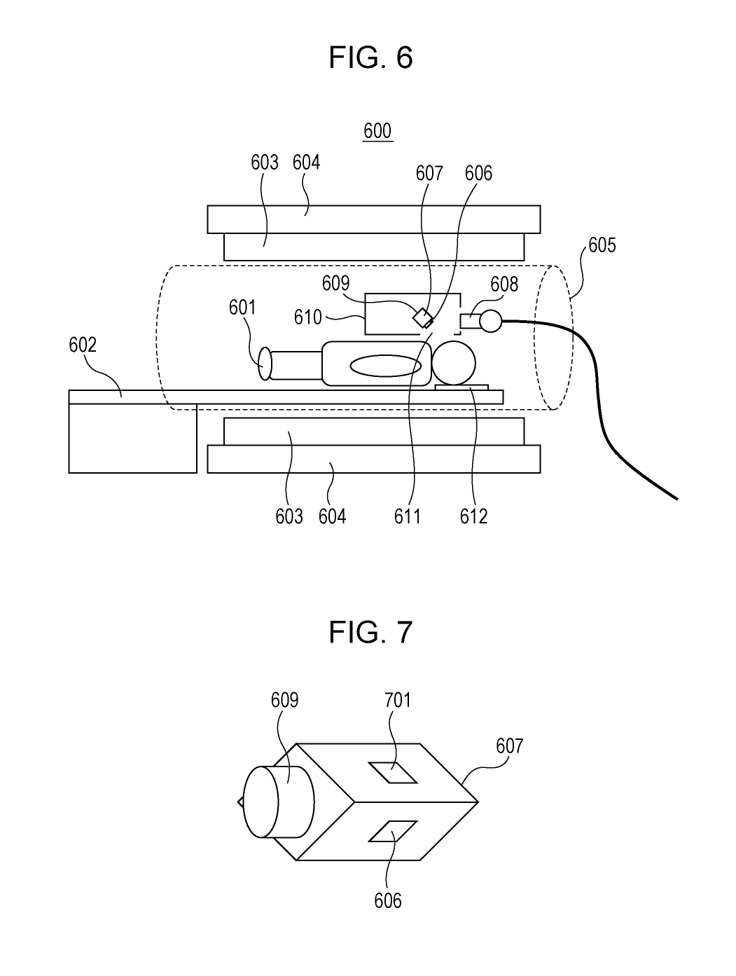

FIG. 6 is a diagram describing the measurement technique used in this example.

In FIG. 6, reference numeral 600 represents an MRI apparatus, reference numeral 601 represents a subject, reference numeral 602 represents a bed of the MRI apparatus 600, reference numeral 603 represents a gradient magnetic field coil, reference numeral 604 represents a superconducting magnet, and reference numeral 605 represents a bore.

In the above-described configuration, the subject 601 was caused to lie down in the interior of the bore 605. In front of the eyes of the subject 601, a sample 606 to be presented as a visual stimulus to the subject 601 was located by a sample holding unit 607.

Also, an optical fiber lighting apparatus 608 for illuminating the sample 606 was located above the head of the subject 601.

The sample holding unit 607 was provided with an ultrasonic motor 609, with which the sample holding unit 607 was controlled, thereby controlling the face of the sample 606 to be presented.

As the ultrasonic motor 609, one driven by a piezoelectric element (electro-mechanical transducer) disclosed in many literatures, such as Japanese Patent Laid-Open No. 3-253272, can be used.

The sample 606 and the sample holding unit 607 were stored in a booth 610 with an aperture 611. In this way, the subject 601 could perceive the sample 606 through the aperture 611.

The lighting apparatus 608 was located so that irradiation light from the lighting apparatus 608 was introduced into the booth 610.

As the sample 606, the sample holding unit 607, the lighting apparatus 608, the ultrasonic motor 609, and the booth 610, ones formed of non-magnetic materials were used. Also, an MR signal detecting coil 612 was located on the back of the head of the subject 601, thereby detecting an electromagnetic signal generated by a change in cerebral blood flow involved in neural activity of the subject 601.

FIG. 7 is an enlarged diagram of the sample holding unit 607 in this example.

The sample holding unit 607, formed of non-magnetic materials, was a rectangular parallelepiped. The sample 606 to be presented to the subject 601 was set on one of the four faces constituting the rectangle.

In this example, a color printed matter was used as the sample 606 to be presented as a visual stimulus to the subject 601. On a face adjacent to the sample 606, a gray control stimulus with the same luminance as the average luminance of the color printed matter was set as a sample 701 serving as a reference.

The ultrasonic motor 609 was attached to a lateral face portion of the sample holding unit 607. The rotation axis of the ultrasonic motor 609 was connected to the center of the lateral face portion of the sample holding unit 607, thereby enabling the sample holding unit 607 to rotate around this rotation axis.

Also, a controller (not shown) for controlling the driving of the ultrasonic motor 609 was located at a place that is in a magnetic shielded room where the MRI apparatus 600 was located and that has the maximum distance from the measuring position of the interior of the body of the MRI apparatus 600.

The ultrasonic motor 609 and the controller were connected by an electromagnetically-shielded control line.

In this way, the ultrasonic motor 609 was capable of driving and controlling, through the controller, the presentation time and the presentation face when selectively presenting the sample 606 or the sample 701 to the subject 601.

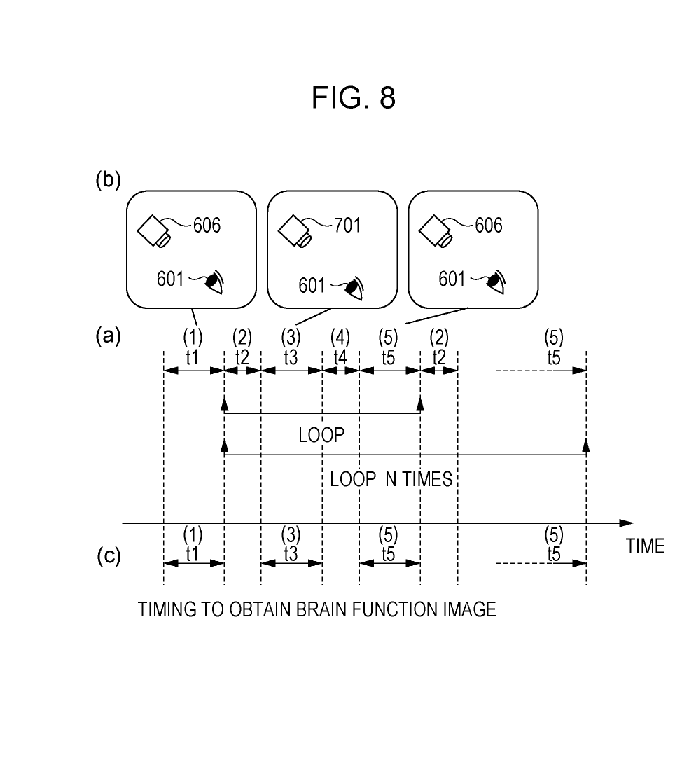

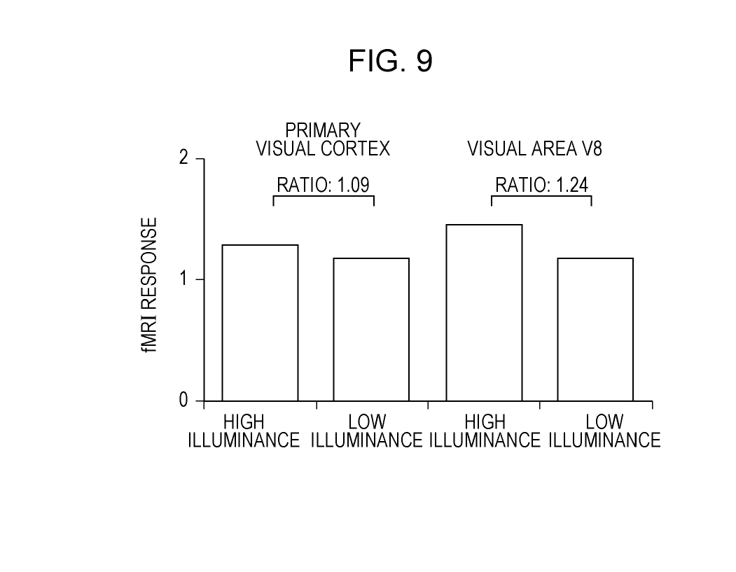

Next, the steps of a brain activity measuring method in this example will be described using FIG. 8.

Firstly, the relationship between the subject 601 and the sample 606 and the reference sample 701 to be presented will be described using FIGS. 8(a) and (b).

FIG. 8(a) shows the order of steps and time intervals between the steps. FIG. 8(b) shows time-series changes in the relationship of the subject 601 with the sample 606 and the reference sample 701.

At first, in step (1), the sample 606 was presented to the subject 601 for a time period t1. Next in step (2), the sample holding unit 607 was rotated 90 degrees in a time period t2 by using the ultrasonic motor 609.

Accordingly, the gray control stimulus serving as the reference sample 701 could be presented.

Next in step (3), the reference sample 701 was presented to the subject 601 for a time period t3. Next in step (4), the sample holding unit 607 was rotated 90 degrees in the opposite direction from step (2) in a time period t4, thereby making it possible to present the sample 606 to the subject 601 again.

Next in step (5), the sample 606 was presented to the subject 601 for a time period t5.

Steps (2) to (5) described above served as one loop, and this loop of driving the ultrasonic motor 609 and changing the face of the sample 606 to be presented was repeated N times in the order of step (2), step (3), step (4), step (5), step (2), . . . and step (5).

On that occasion, the surrounding environment of the subject 601 to which visual stimuli were presented, including the illuminance condition, was maintained to be constant.

In general, in brain measurement based on the method of fMRI, there are multiple techniques for presenting stimuli to a subject (presentation timing). In this example, the technique called “block design” was used.

Block design is a stimulus presenting method including a rest block and a task block. In block design in this example, a state called a “rest block” in which the reference sample 701 was presented to the subject 601 and a state called a “task block” in which the sample 606 was presented to the subject 601 were alternately repeated.

That is, step (3) corresponded to a rest block, and step (5) corresponded to a task block.

Next, using FIG. 8(c), a brain function image obtaining step based on the method of fMRI will be described. Timing to start presenting the sample 606, namely the color printed matter serving as a visual stimulus, and timing to start capturing an image based on the method of fMRI were adjusted to synchronize with each other.

A brain function image in response to presentation of the gray control stimulus serving as the reference sample 701 in the time period t3 in a rest block, i.e., step (3), and a brain function image in response to presentation of the color printed matter serving as the sample 606 in the time period t5 in a task block, i.e., step (5), were obtained.

In this example, the loop from step (2) to step (5) was repeated N times. Thus, N brain function images in task blocks and N brain function images in rest blocks were obtained.

Noise was removed from these images by performing image processing such as equalization, and finally the brain function images in task blocks and in rest blocks were compared with each other, thereby obtaining a brain function image when the subject 601 perceived the visibility of colors of the color printed matter.

In the brain function image obtaining step in this example, the ultrasonic motor 609 was rotated in step (2) and step (4) to change the sample to be presented to the subject 601.

However, it is preferable to make the brain function image obtaining step based on the method of fMRI not to obtain brain function images in these time periods t2 and t4.

There are two reasons for this. One reason is that, in this example, because the tilt of the sample holding unit 607 was changed in the time periods t2 and t4, brain function images obtained in these time periods may contain both images in the task and rest blocks.

The other reason is that, because the ultrasonic motor 609 was driven, a very small electromagnetic wave generated thereby may deteriorate the brain function images.

By stopping obtaining brain function images in time periods in which the ultrasonic motor 609 is driven, it becomes possible to prevent noise caused by driving the ultrasonic motor 609 from being superimposed on image data used in analysis after the measurement.

There are some types of MRI apparatuses incapable of stopping obtaining brain function images only in the time periods t2 and t4 as described above.

In this case, after brain function images are obtained for the entire measurement period, brain function images obtained in the time periods t2 and t4 are not used, and only image data obtained in the time periods t3 and t5 is analyzed, thereby obtaining a desired brain function image.

The illuminance conditions set in this example were a low illuminance condition (1000 lux) and a high illuminance condition (10000 lux). In setting the illuminance conditions, attention was paid not to change physical properties of light other than the illuminance, such as physical property values including the color temperature and chromaticity.

For the illuminance conditions, the brain activity of the subject 601 in response to presentation of a color printed matter serving as a visual stimulus was measured using the above-described brain activity measuring technique and block design.

In the visual cortex of the cerebral cortex, brain activity in the primary visual cortex and visual area V8 was measured.

From the measured and obtained brain activity information, the ratios of brain activity data under the illuminance conditions in the primary visual cortex and visual area V8 were calculated.

Specifically, luminance information in the primary visual cortex and visual area V8 was calculated from captured fMRI images, and time-series changes in the luminance information served as time-series changes in brain activity data.

Further, the amplitude values of the waveforms of the time-series changes were obtained.

FIG. 9 is a bar graph showing the amplitude values of brain activity data under the low illuminance condition and the high illuminance condition in the visual cortical areas.

Using the result shown in FIG. 9, the ratios of amplitude values in the visual cortical areas involved in changing the illuminance condition were calculated. Using the ratio (ratio 3) between the calculated ratio in visual area V8 (ratio 2) and the calculated ratio in the primary visual cortex (ratio 1) as an index (ratio 3 is ratio 2/ratio 1), information relating to the visibility of colors when the subject 601 perceived the color printed matter in an environment under different illuminance conditions was obtained.

In evaluation of perception relating to the visibility of colors in an environment under different illuminance conditions, a comparison was made with a preset threshold. The threshold in this example was set as described below.

For ten subjects, fMRI images in response to presentation of the color printed matter were obtained under different illuminance conditions by using the same brain activity measuring apparatus and the same brain activity measuring technique as those in this example.

Luminance information relating to the primary visual cortex and visual area V8 in the obtained fMRI images was calculated for the illuminance conditions.

Time-series changes in luminance served as time-series changes in brain activity data, and further the amplitude values of the waveforms of the time-series changes in brain activity data were obtained. The above calculation was repeated to obtain the amplitude values of brain activity data of ten subjects, and a t-test was conducted to determine whether there was a significant difference in brain activity data in the visual cortical areas under different illuminance conditions.

The amplitude values in the primary visual cortex in response to presentation of the color printed matter under the two illuminance conditions were 1.17 under the low illuminance condition and 1.28 under the high illuminance condition. As a result of a t-test, no significant difference was determined in the primary visual cortex.

Thus, the ratio between the amplitude values under the two illuminance conditions was obtained, and this ratio was set as a threshold in the primary visual cortex. The ratio between the amplitude values was 1.28/1.17=1.09. Therefore, the ratio to be set was within a range in which the ratio between the calculated amplitude values was from 0.9 to 1.1.

In contrast, the amplitude values in visual area V8 in response to presentation of the color printed matter under the two illuminance conditions were 1.17 under the low illuminance condition and 1.45 under the high illuminance condition. As a result of a t-test, a significant difference was determined in visual area V8. Thus, the ratio between the amplitude values under the two illuminance conditions was obtained, and this ratio was set as a threshold in visual area V8. The ratio between the amplitude values was 1.45/1.17=1.24.

Therefore, the ratio to be set was such that the ratio between the calculated amplitude values was greater than or equal to 1.2.

Using these ratios, evaluation was performed to determine under which of the illuminance conditions a subject to which the color printed matter was presented perceived colors more colorfully.

That is, when the ratio between the amplitude values in the primary visual cortex was within the range from greater than or equal to 0.9 to less than or equal to 1.1 and when the ratio between the amplitude values in visual area V8 was greater than or equal to 1.2, it was evaluated that the subject perceived colors more colorfully under the high illuminance condition.

According to this example, since the method of fMRI was used as the brain activity measuring method, brain activity in each area of the visual cortex of the cerebral cortex can be measured at a high spatial resolution, thereby obtaining a highly accurate evaluation result.

For example, the configuration discussed in this example is preferable in measuring brain activity in a specific area of the visual cortex of the cerebral cortex, such as visual area V8.

### Second Example

In a second example, brain activity in the primary visual cortex and in visual area V8, which is the color vision related area, in response to presentation of a color printed matter and a monochrome printed matter to a subject under the same illuminance condition was measured, and, using the obtained brain activity data, the subject's perception relating to the visibility of colors of the color printed matter was evaluated.

Also in this example, as in the first example, the MRI apparatus and the method of fMRI were used.

The illuminance condition set in this example was 10000 lux. In setting the illuminance condition, attention was paid not to change physical properties of light other than the illuminance, such as physical property values including the color temperature and chromaticity. Under the set illuminance condition, the brain activity of a subject in response to presentation of a color printed matter and a monochrome printed matter serving as visual stimuli was measured using the above-described brain activity measuring technique and block design, as in the first example.

From the measured and obtained brain activity information, the ratios of brain activity data in response to each of the visual stimuli in the primary visual cortex and visual area V8 were calculated. As in the first example, a comparison was made with a preset threshold.

The amplitude values in the primary visual cortex were 1.28 in response to the color printed matter and 1.16 in response to the monochrome printed matter. As a result of a t-test, no significant difference was determined in the primary visual cortex.

Thus, the ratio between the amplitude values was obtained, and this ratio was set as a threshold in the primary visual cortex. The ratio between the amplitude values was 1.28/1.16=1.10. Therefore, the ratio to be set was within a range in which the ratio between the calculated amplitude values was from 0.9 to 1.1.

In contrast, the amplitude values in visual area V8 were 1.45 in response to the color printed matter and 1.12 in response to the monochrome printed matter. As a result of a t-test, a significant difference was determined in visual area V8. Thus, the ratio between the amplitude values was obtained, and this ratio was set as a threshold in visual area V8. The ratio between the amplitude values was 1.45/1.17=1.24.

Therefore, the ratio to be set was such that the ratio between the calculated amplitude values was greater than or equal to 1.2.

Using these ratios, evaluation was performed to determine in response to which of the visual stimuli a subject perceived colors more colorfully.

That is, when the ratio between the amplitude values in the primary visual cortex was within the range from greater than or equal to 0.9 to less than or equal to 1.1 and when the ratio between the amplitude values in visual area V8 was greater than or equal to 1.2, it was evaluated that the subject perceived colors more colorfully in response to the color printed matter.

While the present disclosure has been described with reference to exemplary embodiments, it is to be understood that these embodiments are not seen to be limiting. The scope of the following claims is to be accorded the broadest interpretation so as to encompass all such modifications and equivalent structures and functions.

This application claims the benefit of Japanese Patent Application No. 2012-182962 filed Aug. 22, 2012, which is hereby incorporated by reference herein in its entirety.

What is claimed is:

1. An information obtaining method of obtaining brain activity information from a living body to which different visual stimuli are given, comprising:
(1) presenting a first visual stimulus to the living body;
(2) obtaining brain activity data in the primary visual cortex of the living body and brain activity data in a color vision related area of the living body during presentation of the first visual stimulus;
(3) presenting a second visual stimulus different from the first visual stimulus to the living body;
(4) obtaining brain activity data in the primary visual cortex and brain activity data in the color vision related area during presentation of the second visual stimulus; and
(5) obtaining brain activity information on the basis of the ratio between the brain activity data in the primary visual cortex, which is obtained in (2), and the brain activity data in the primary visual cortex, which is obtained in (4), and the ratio between the brain activity data in the color vision related area, which is obtained in (2), and the brain activity data in the color vision related area, which is obtained in (4).

2. The information obtaining method according to claim 1, wherein the color vision related area is visual area V4 or visual area V8.

3. The information obtaining method according to claim 1,
wherein (1) includes presenting an object with a chromatic color irradiated with light with a first illuminance, and wherein (3) includes presenting the object with the chromatic color irradiated with light with a second illuminance different from the first illuminance.

4. The information obtaining method according to claim 3, further comprising, after (1) and before (3), presenting an object with an achromatic color generated by removing hue and saturation from the object with the chromatic color.

5. The information obtaining method according to claim 1, further comprising, after (1) and before (3), returning brain activity in the primary visual cortex and the color vision related area of the living body to a steady state.

6. The information obtaining method according to claim 1, wherein the brain activity information is information relating to colorfulness of colors perceived by the living body.

7. The information obtaining method according to claim 1, wherein, in (2) and (4), data relating to at least one selected from a change in action potential of the brain of the living body, a change in an electromagnetic field caused by the change in action potential, and a change in cerebral blood flow caused by the change in action potential is obtained as the brain activity data.

8. The information obtaining method according to claim 1, wherein luminance information of an fMRI image is obtained in (2) and (4).

9. A method of evaluating which of two objects, presented to a subject, has colors that are perceived by the subject to be more colorful than the other, comprising:
(1) presenting a first object to the subject;
(2) obtaining an fMRI image of the primary visual cortex of the subject and an fMRI image of visual area V4 or visual area V8 of the subject during presentation of the first object;
(3) presenting a second object different from the first object to the subject;
(4) obtaining an fMRI image of the primary visual cortex and an fMRI image of visual area V4 or visual area V8 during presentation of the second object; and
(5) evaluating that the subject perceives colors of the second object to be more colorful in the case where luminance of the fMRI image of the primary visual cortex, which is obtained in (4), is in a range from greater than or equal to 0.9 times to less than or equal to 1.1 times luminance of the fMRI image of the primary visual cortex, which is obtained in (2), and luminance of the fMRI image of visual area V4 or visual area V8, which is obtained in (4), is greater than or equal to 1.2 times luminance of the fMRI image of visual area V4 or visual area V8, which is obtained in (2).

10. The evaluation method according to claim 9, wherein the first object is an object irradiated with light with a first illuminance, and the second object is the object irradiated with light with a second illuminance different from the first illuminance.
